(19) United States
(12) Patent Application Publication
(10) Pub. No.: US 2015/0007831 A1
(43) Pub. Date: Jan. 8, 2015

(54) Oral Appliance and Tray

(71) Applicant: Kevin Barrett, Los Angeles, CA (US)

(72) Inventors: Kevin Barrett, Pacific Palisades, CA (US); Tim L. Frink, Fountain Valley, CA (US); Carl TenBrink, Huntington Beach, CA (US)

(21) Appl. No.: 13/935,412

(22) Filed: Jul. 3, 2013

(51) Int. Cl. A61F 5/00

(52) U.S. Cl. CPC A61F 5/0006
USPC 128/861

(57) ABSTRACT

An oral appliance and accompanying tray is provided, where the oral appliance is fitted to the posterior teeth of the upper jaw and a connecting portion extends between the left and right sides of the mouth, with the connecting portion custom-fitted to contact the palate at or near the palatal nerve to reduce appetite. The present tray has a compressible member configured to closely form the connecting portion to the palatal arch. Further, the tray has flexible ends that are configured to bend up towards the gums and guide the oral appliance to fit the buccal side of the teeth. The tray may include a series of protrusions on the occlusal landing areas to prevent the user from biting through the oral appliance.

### BACKGROUND

The present invention relates to an oral appliance and tray for fitting the oral appliance, and more particularly, to an oral appliance that fits over the posterior teeth of the upper jaw and a tray for fitting the oral appliance.

The wearing of oral appliances, such as retainers or clear teeth aligners (as made by INVISALIGN), has been associated with reduced caloric intake and resulting weight loss. It is thought that the oral appliances increase the inconvenience of eating just slightly, due to the user having to take the appliance out in order to eat and perhaps due to the sensation of the appliance in the mouth. However, these oral appliances are used for limited and specific purposes which are related to dental health. As such, they must be prescribed and fitted by a dentist specifically to correct and improve dental health. Thus, if a person does not have a specific need for or does not want a dental appliance, then the person cannot take advantage of the potential weight loss benefits. Further, an average person may not care to have an oral appliance that is visible when they talk or smile.

Additionally, there is great difficulty in fitting oral appliances outside a dentist's or orthodontist's office, due to the variability in mouth shapes and errors introduced by the user self-fitting an oral appliance at home. Often, ill-fitting oral appliances do not properly grip the teeth, and may dislodge during use or cause undue discomfort.

Therefore, what is needed is a non-prescription oral appliance that provides the weight loss benefits of a corrective appliance, without being visible to others. What is further needed is a tray for aiding the fitting an oral appliance at home, which can accommodate various mouth shapes and corrects much of the user-induced error.

### SUMMARY

A tray for fitting an oral appliance within a mouth of a user is provided. The mouth has a palatal arch and an upper jaw with a plurality of left posterior teeth and a plurality of right posterior teeth. The tray preferably includes a left occlusal landing area configured to receive the left posterior teeth and a right occlusal landing area configured to receive the right posterior teeth. A connecting portion spans between the left occlusal landing area and the right occlusal landing area. A compressible member is located on the connecting portion, and may optionally be arch-shaped. In use, the oral appliance is overlaid atop the left occlusal landing area, the right occlusal landing area, and the compressible member, which are cooperatively shaped to closely fit the oral appliance. The oral appliance is configured to be positioned between the compressible member and the palatal arch while in a moldable state. Upon biting down, the compressible member is compressed by the palatal arch, with the oral appliance being formed against the palatal arch.

The compressible member may optionally be an arch or arch-shaped. Optionally, a radius of the arch is configured to be smaller than the radius of the palatal arch of the user and/or a height of the arch is configured to be taller than the height of the palatal arch of the user, such that compression of the compressible member causes the radius of the arch to deform to substantially the contours of the palatal arch. Optionally, the compressible member may have a groove shaped to grip the oral appliance.

Optionally, the left occlusal landing area has a left upper surface and a left protrusion extending from the left upper surface and the right occlusal landing area has a right upper surface and a right protrusion extending from the right upper surface. Upon the user biting down upon the oral appliance while in a moldable state, the left protrusion is configured to prevent occlusal surfaces of the left posterior teeth from contacting the left upper surface, and the right protrusion is configured to prevent occlusal surfaces of the right posterior teeth from contacting the right upper surface.

Optionally, a left flexible portion extends from a left buccal side of the left occlusal landing area and a right flexible portion extends from a right buccal side of the right occlusal landing area. Upon the user biting down upon the oral appliance while in a moldable state, the left flexible portion is configured to be flexed upwardly by contact with the left cheek and flexed towards the left buccal sides of the left posterior teeth, and the right flexible portion is configured to be flexed upwardly by contact with the right cheek and flexed towards the right buccal sides of the right posterior teeth.

In another embodiment, the tray includes an occlusal landing area configured to receive the posterior teeth and a flexible portion extending from the buccal side of the occlusal landing area. The occlusal landing area is configured to support a portion of the oral appliance, where the portion is overlaid atop the occlusal landing area and the flexible portion. Upon the user biting down upon the oral appliance while in a moldable state, the flexible portion is configured to be flexed upwardly by contact with the cheek and flexed towards the buccal sides of the posterior teeth.

Optionally, the flexing of the flexible portion aids in forming the oral appliance against the buccal sides of the posterior teeth such that a terminal portion of the oral appliance extends over the height of contours of the posterior teeth and is formed to at least a portion of the infrabulge areas.

In yet another embodiment, the tray includes an occlusal landing area with an upper surface and a protrusion extending from the upper surface, where the occlusal landing area is configured to receive the posterior teeth. The occlusal landing area is configured to support a portion of the oral appliance, where the portion is overlaid atop the upper surface. Upon the user biting down upon the oral appliance while in a moldable state, the protrusion on the upper surface is configured to prevent the occlusal surfaces of the posterior teeth from contacting the upper surface of the occlusal landing area. Optionally, the protrusion may be a ridge and there may be a plurality of protrusions.

### BRIEF DESCRIPTION OF THE SEVERAL VIEWS OF THE DRAWINGS

FIG. 1 is a front exploded perspective view of the preferred embodiment of the present oral appliance and tray;

FIG. 2 is a back exploded perspective view of the preferred embodiment of the present oral appliance and tray

FIG. 3 is a bottom exploded perspective view of the preferred embodiment of the present oral appliance and tray

FIGS. 4A-B are exploded plan views of the front and side respectively;

FIGS. 5A-B are assembled plan views of the front and side respectively;

FIGS. 6A-C are assembled perspective and plan views of the present tray with the oral appliance attached;

FIGS. 7A-C are perspective and plan views of the base tray of the present tray;

FIGS. 8A-E are perspective and plan views of the present oral appliance separate from the tray;

FIGS. 9A-E are perspective and plan views of the present compressible member separate from the base tray;

FIGS. 10A-B a cross-sectional and back plan view of the present oral appliance attached to the tray;

FIG. 11 is a view of the present oral appliance fitted to the posterior teeth of an upper jaw and contacting the palatal arch;

FIG. 12 is a cross-sectional view of the present oral appliance being fitting to the posterior teeth of the upper jaw by the tray; and

FIG. 13A-B are a plan and a cross-sectional view of an alternate embodiment of the present oral appliance and tray.

### LISTING OF REFERENCE NUMERALS OF FIRST-PREFERRED EMBODIMENT

tray 20

tray base 21

oral appliance 22

compressible member 24

right occlusal landing area 26

left occlusal landing area 28

connecting portion 30

right protrusion 32

left protrusion 34

right upper surface 36

left upper surface 38

right flexible portion 40

left flexible portion 42

right buccal side 44

left buccal side 46

groove 48

right terminal portion 50

left terminal portion 51

handle 52

front teeth landing area 54

stop 56

tongue clearance 58

brace 60

depression 62

right occlusal portion 64

left occlusal portion 66

palatal portion 68

underside 70

hard pad 72

lower occlusal surface 74

arch radius r

arch height h

mouth 200

upper jaw 202

right posterior teeth 206

left posterior teeth 208

buccal side 210

occlusal surface 212

infrabulge area 214

height of contour 216

front teeth 218

gum 220

suprabulge area 222

cheek force F

### DESCRIPTION OF THE PREFERRED EMBODIMENTS

The detailed descriptions set forth below in connection with the appended drawings are intended as a description of embodiments of the invention, and is not intended to represent the only forms in which the present invention may be constructed and/or utilized. The descriptions set forth the structure and the sequence of steps for constructing and operating the invention in connection with the illustrated embodiments. It is to be understood, however, that the same or equivalent structures and steps may be accomplished by different embodiments that are also intended to be encompassed within the spirit and scope of the invention.

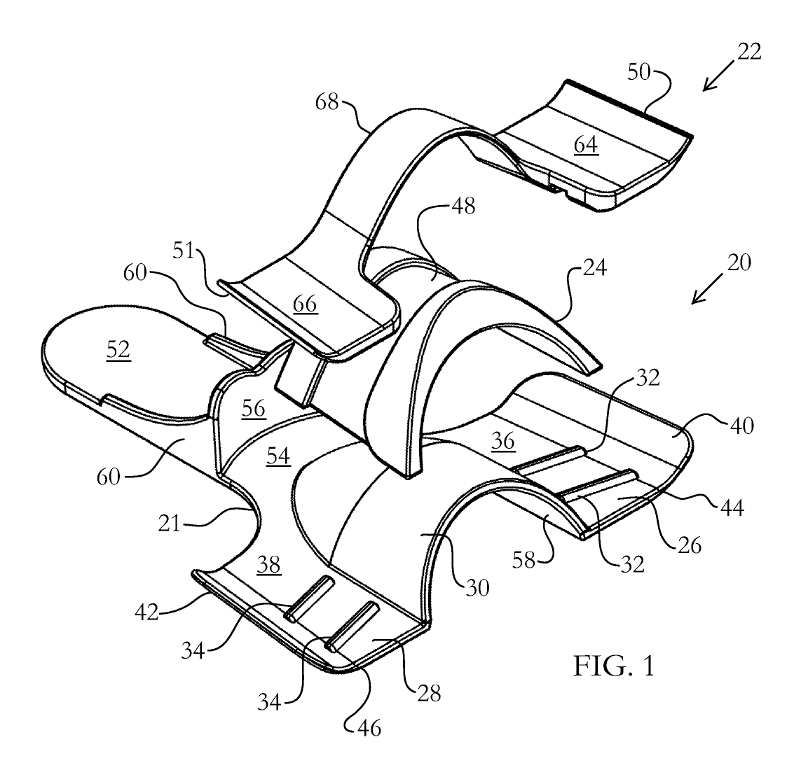

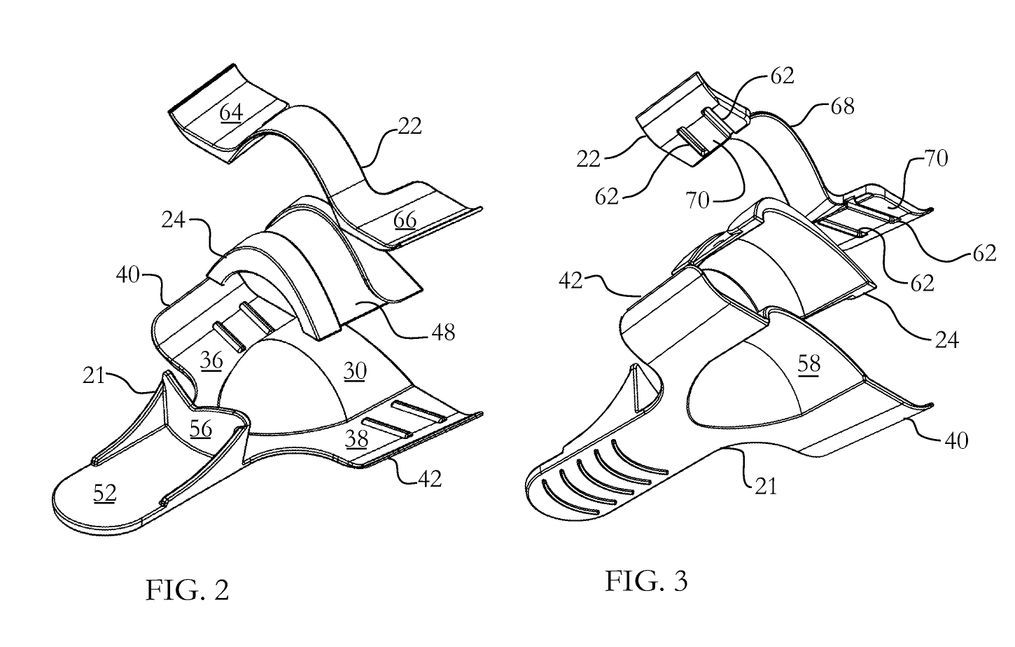

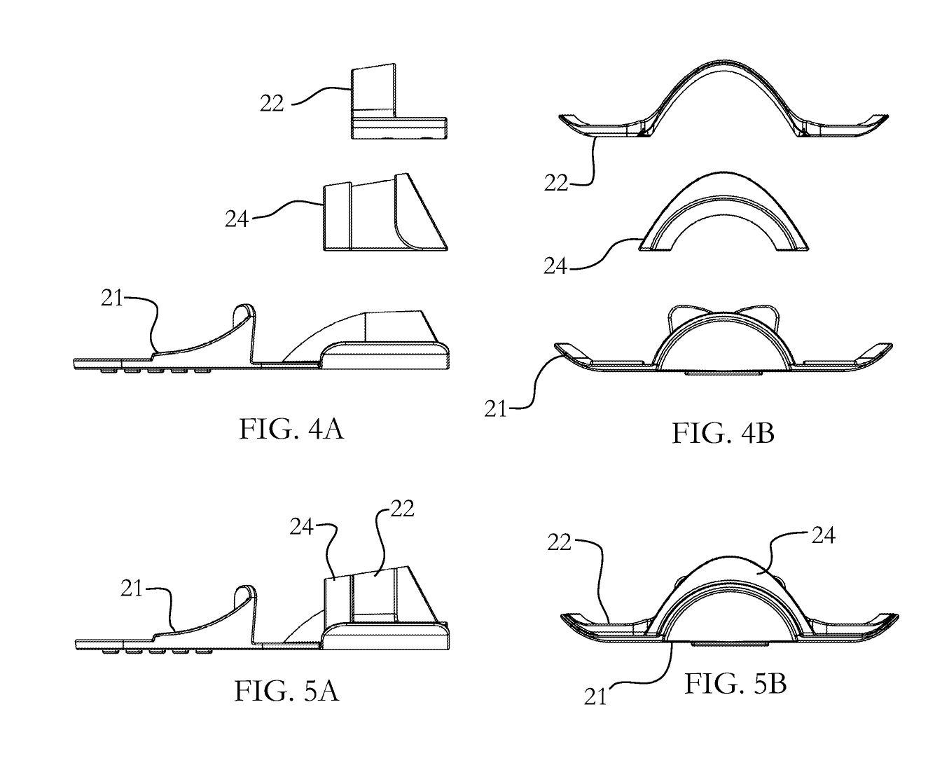

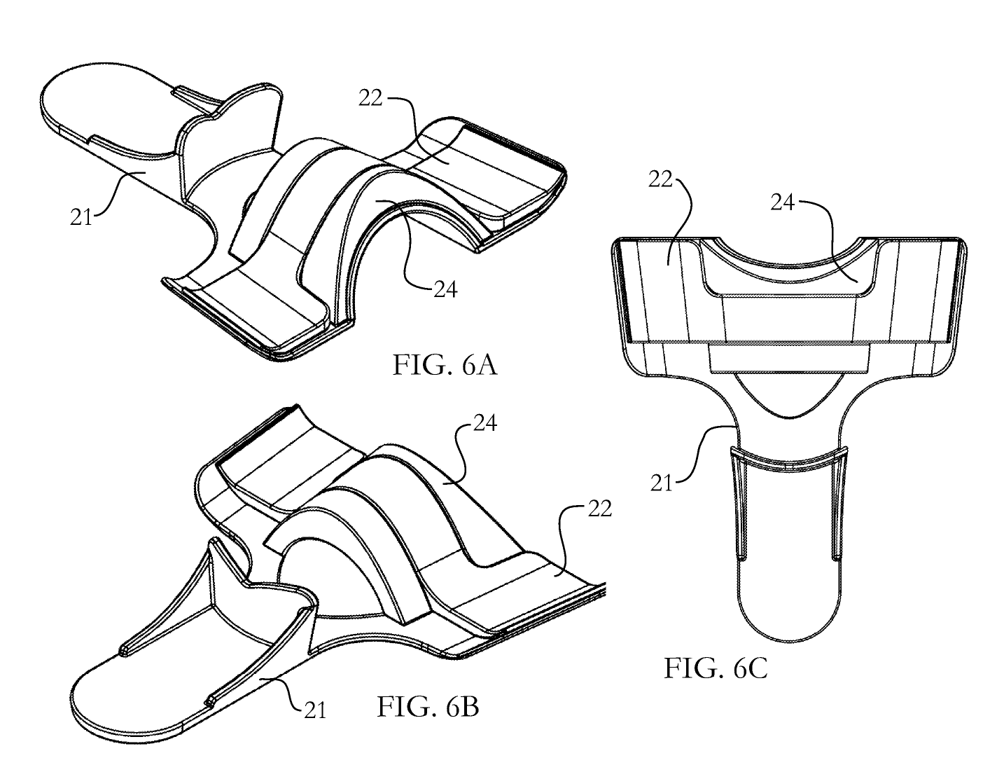

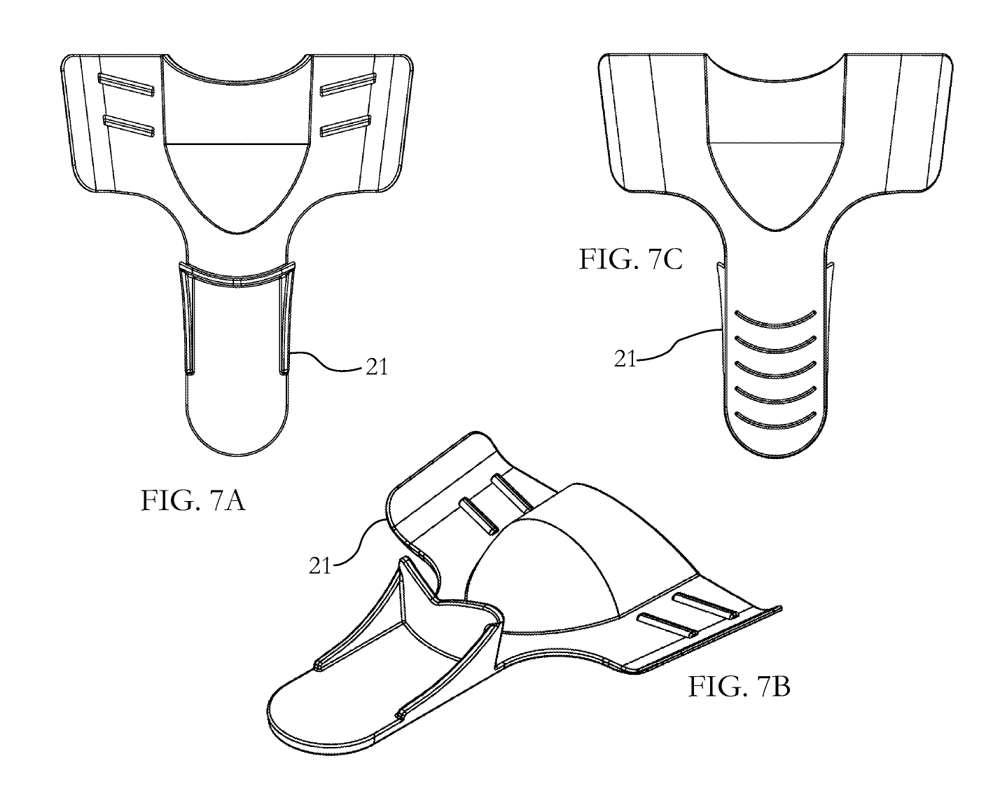

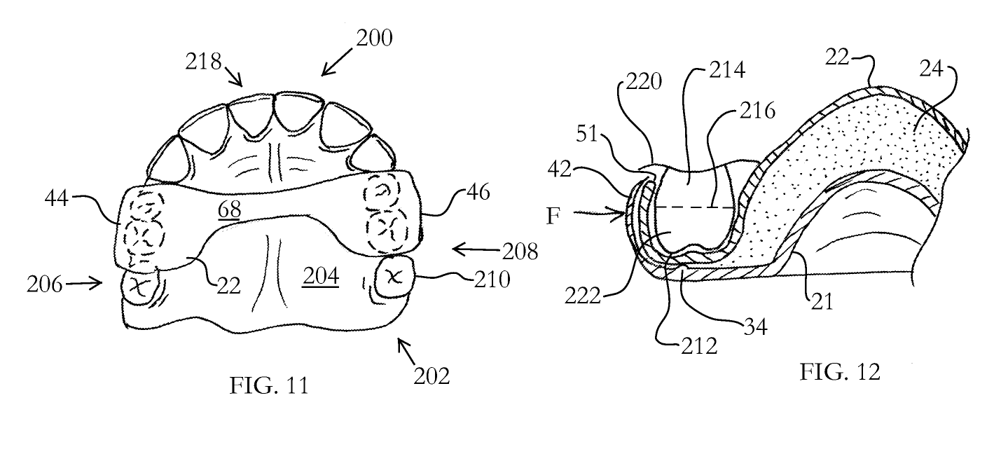

Looking at FIGS. 1-12, the present tray (20) and oral appliance (22) is shown. The tray (20) is configured to hold the oral appliance (22) while the oral appliance (22) is being prepared, fitted, and cured within the mouth. In the illustrated example embodiment, the tray (20) has a right occlusal landing area (26) connected to a left occlusal landing area (28). The connection between the right occlusal landing area (26) and the left occlusal landing area (28) can be made through a unitary plastic molded design, where the two landing areas are molded to a common handle (52) or other appropriate portion of the tray base (21).

As illustrated, the tray base (21) is injection molded from a unitary piece of plastic; and the right occlusal landing area (26) and the left occlusal landing area (28) are connected through the structure of the handle (52) at the front teeth landing area (54) and through the connecting portion (30). The connection between the landing areas (36, 38) can be made through either means, such as just the connecting portion (30). Here, the connecting portion (30) is generally arch-shaped to provide a tongue clearance (58) when the tray (20) is in the mouth. The front teeth landing area (54) and the connecting portion (30) provide appropriate structure to hold the right occlusal landing area (26) and the left occlusal landing area (28) substantially stationary relative to one another, although some flexing may be permitted. To increase rigidity and strength of the handle (52), a vertical stop (56) has a brace (60) integrally molded on both sides.

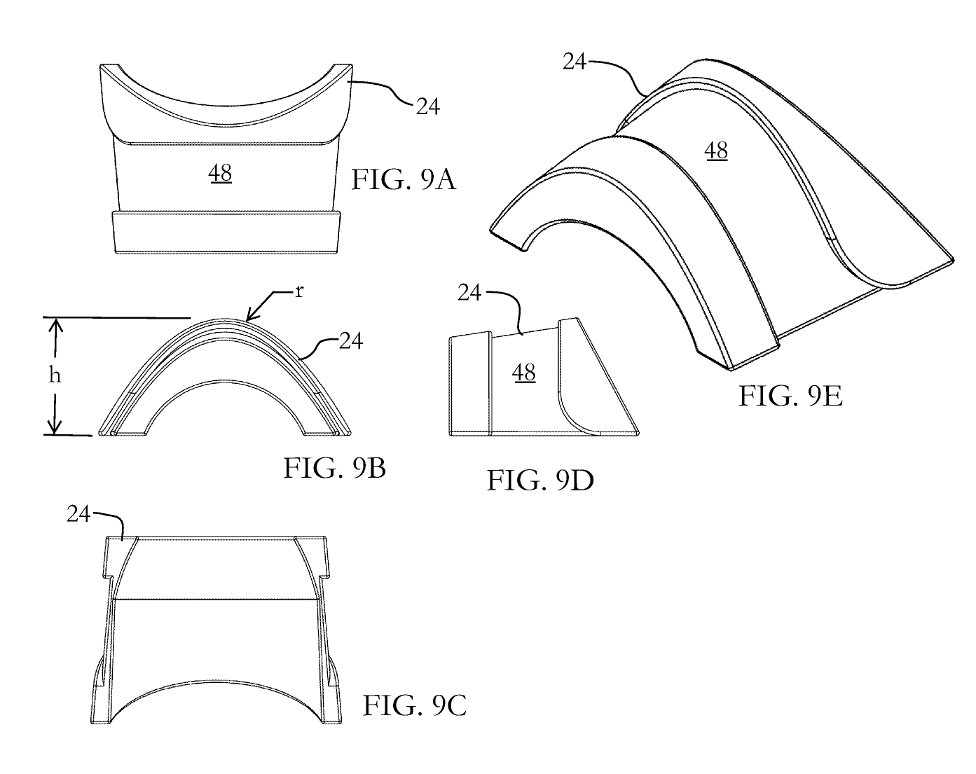

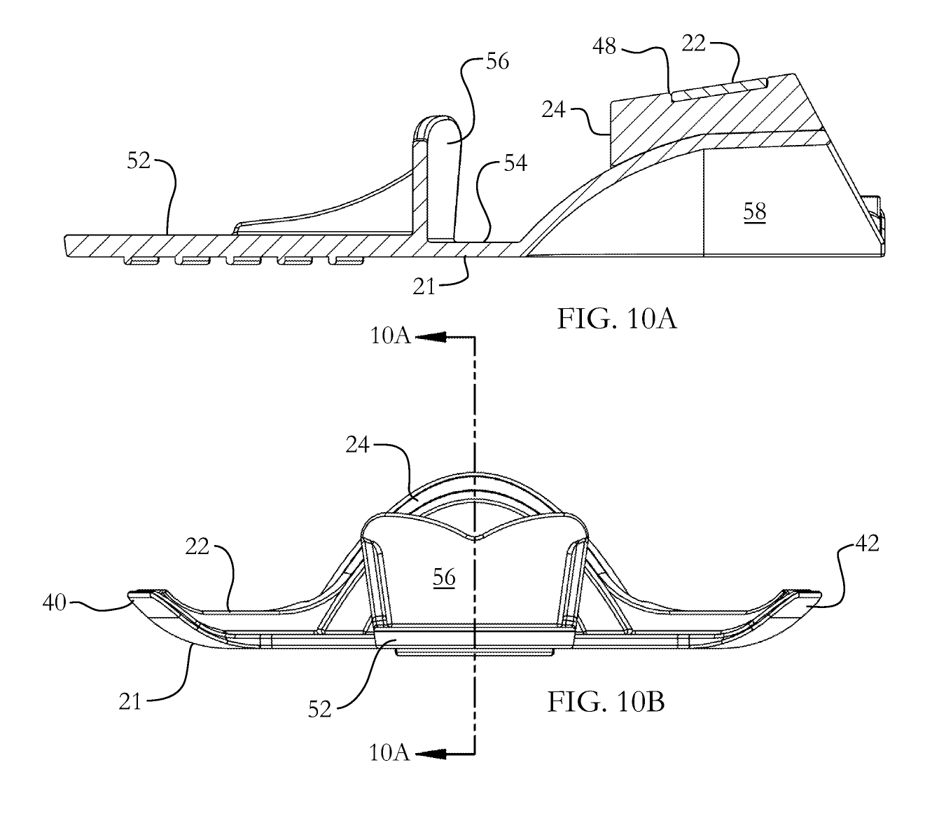

Situated atop the connecting portion (30) is a compressible member (24). The compressible member (24) can be made of an elastically deformable material or of a material that permanently deforms when a deforming force is applied. A preferred material is a foam rubber or other similar material, which can generally conform to the shape of the user's palatal arch (204). The compressible member (30) is shown as having a generally arch shape. Other shapes for the compressible member (24) may be used, so long as the shape compresses to conform generally to the palatal arch (204). The arch shape is chosen to more closely match the average user's palatal arch (204) at a section taken from the right posterior teeth (206) to the left posterior teeth (208) and generally parallel to the coronal plane. Further, as can be more clearly seen in FIGS. 9D and 10A, the compressible member (30) is generally shaped to fit the average palate when viewed from a section transverse to the coronal plane (parallel to the sagittal plane). Thus, the general shape of the compressible member (30) at the surface which will be facing towards the user's palatal arch (204) is the general shape of a surface section of a cone. This shape generally follows the palatal arch (204), which is arched and angled.

The compressible member (24) has a groove (48) formed on the surface which will be facing towards the user's palatal arch (204) when in the mouth. The groove (48) is shaped to receive the palatal portion (68) of the oral appliance (22). The groove (48) may be configured to hold the oral appliance (22) through a frictional engagement or interference engagement. With a frictional engagement, the groove (68) is slightly narrower than the width of the palatal portion (68) of the oral appliance (22), so that the palatal portion (68) fits and is held snuggly within the groove (48). In an example of an interference engagement, the walls of the groove (48) are undercut so that the palatal portion (68) is trapped within the groove (48) until the user detaches the oral appliance (22) by application of sufficient pulling force. A food safe adhesive may also be used to temporarily attach the oral appliance (22) to the tray (20).

The oral appliance (22) is roughly shaped to fit across the upper jaw (202), with a palatal portion (68) spanning between a right occlusal portion (64) and a left occlusal portion (66). A right terminal portion (50) extends from the right occlusal portion (64); and a left terminal portion (51) extends from the left occlusal portion (66). The right occlusal portion (64) is configured to receive the right posterior teeth (206) under biting pressure, such that the occlusal surfaces (212) of the teeth contact the right occlusal portion (64) and act to form the oral appliance, as will be discussed further below. Similarly, the left occlusal portion (66) is configured to receive the left posterior teeth (208) under biting pressure. The right terminal portion (50) and the left terminal portion (51) are curved upwardly to encourage the terminal portion (50, 51) to curl around and form to the buccal sides (210) of the posterior teeth (206, 208) upon being contacted and further bent by the cheeks.

The combination of wide occlusal landing areas (36, 38), the compressible member (24), and optionally, the flexible portions (40, 42), permit the tray (20) to fit within a wide variety of mouth sizes and shapes. Thus, the present tray (20) can be made in one size or just a few sizes, yet fit a broad variety of users. The wide landing areas (36, 38) can accommodate users with narrow upper jaws, when measured from the right posterior teeth (206) to the left posterior teeth (208), to users with wide upper jaws. When the tray (20) is fitted within a narrow upper jaw, the posterior teeth (206, 208) will be situated close to the connecting portion (30); and with a wide upper jaw the posterior teeth (206, 208) will be situated closer to the flexible portions (40, 42). Further, the present tray (20) can be fitted to users with high or low palatal arches (204). For example, if the user has a low palatal arch (204) and a narrow upper jaw (202), the compressible member (24) will be substantially compressed and reduced in size to fit the user's small mouth. A larger mouth would compress the compressible member (24) to a lesser degree or not at all. For a wide or narrow upper jaw (202), the flexible portions (40, 42) are configured to bend upwardly towards the gums of the upper jaw (202) under the pressure form the user's cheeks.

The front teeth landing area (54) provides a comfortable area for the front teeth and helps to align the tray (20) within the mouth, while the stop (56) rests against the front teeth or lips to properly locate the tray (20) and prevent the tray (20) from being inserted too far into the mouth. The front teeth landing area (54) provides a well centered bite or occlusion to insure the right and left side of the upper jaw (202) will land evenly on their respective occlusal portions (64, 66).

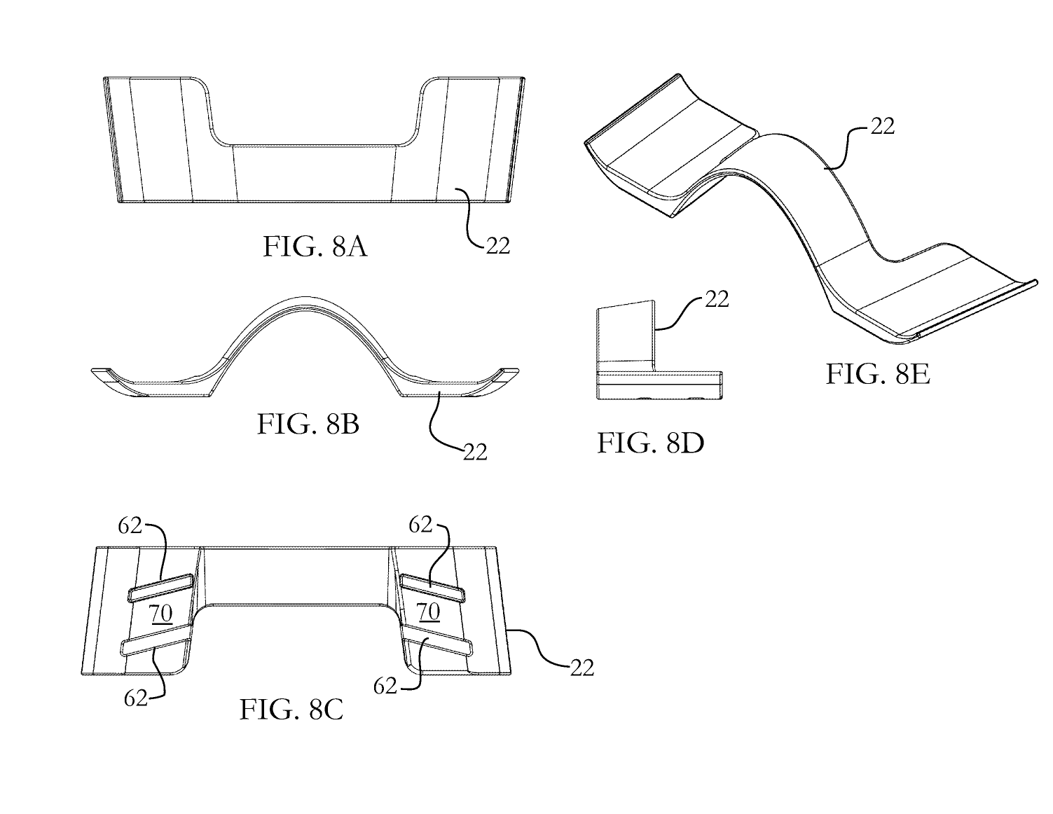

Referring to FIGS. 8A-E, the oral appliance (22) is preferably made from a thermosoftening plastic (such as HYDROPLASTIC, available from TAK SYSTEMS), which can be softened to a moldable state by submersion in hot water. Preferably, the tray base (21) and the compressible member (24) are made from a plastic material that can withstand exposure to substantially higher temperatures than the oral appliance (22) without softening or losing structural integrity. On the underside (70) of the oral appliance (22) are depressions (62) that are configured to receive the protrusions (32, 34) therein. In the illustrated example, the protrusions (32, 34) are elongated half-round ridges, with a 3/32 inch radius. The depressions (62) are similarly shaped and sized, so that the protrusions (32, 34) fit within the depressions (62) when the oral appliance (22) is fitted to the tray (20). The protrusions (32, 34) engaged within the depressions (62) aid in locating and holding the oral appliance (22) on the tray (20) while inserting the assembly into the mouth and while biting down on the occlusal portions (64, 66).

In one example method of using the tray (20) and oral appliance (22), the user attaches the oral appliance (22) to the tray (20) or the tray (22) can be attached during manufacturing. Water is heated approximately to a boil and is poured into a container or left in the container used for boiling. Thus, the water will cool to under the boiling temperature and preferably will have a temperature between approximately 155° F. and 180° F. Some materials selected may require a slightly lower range between approximately 120° F. and 160° F. Of course an exact temperature can be obtained and maintained by heating the water within a thermostat controlled container. The user completely submerges the oral appliance (22) within the hot water by holding the handle (52) of the tray (20) and dipping the assembly, with the handle (52) remaining above the water. The oral appliance (22) should remain within the hot water until sufficiently softened to permit molding within the mouth. The water should not be so hot that user discomfort or burns result. The oral appliance (22) thermosoftening plastic material can be chosen to turn color when sufficiently heated, such as turning from opaque to translucent. It has been experimentally found that heating the oral appliance (22) in 155° F. to 180° F. water for approximately 1.5 to 3 minutes sufficiently prepares the oral appliance (22) for molding.

The user removes the assembly from the water, then immediately places the tray (20) and oral appliance (22) in the mouth (200). Accurate positioning within the mouth is achieved by the user positioning the handle (52) straight ahead and inserting the assembly into the mouth until reaching wall of the stop (56). The user then bites down on the assembly, with the front teeth (218) positioned at, but not necessarily touching, the front teeth landing area (54). The occlusal surfaces (212) of the right posterior teeth (206) will contact the right occlusal portion (64) of the oral appliance (22). The occlusal surfaces (212) of the left posterior teeth (208) will contact the left occlusal portion (66) of the oral appliance (22). The user will then bite down to compress and form the softened oral appliance (22) between the tray (20) and the occlusal surfaces (212). The user will hold the cheeks close to the buccal side (210) of the posterior teeth (206, 208), which will bend the flexible portions (40, 42) around the buccal side (210) and towards the gums (220). The flexible portions (40, 42) are not required to bend completely up and against the buccal sides (210). The bending should be sufficient to guide the softened material of the terminal portions (50, 51) towards the buccal sides (210).

Although it is not necessary, it is preferable that the terminal portions (50, 51) be pushed against the buccal sides (210) of the posterior teeth (206, 208). The terminal portions will preferably cover the suprabulge areas (222) and extend over the height of contour (216) and into the infrabulge area (214) of each of the affected posterior teeth (206, 208). Thus, preferably, the terminal portions (50, 51) will extend over the buccal sides (210) to terminate at a region gingival to the height of contour (216) (the tooth's point of greatest circumference).

As the user bites down, the protrusions (32, 34) prevent the occlusal surfaces (212) from contacting the upper surfaces (36, 38). The soft material of the occlusal portions (64, 66) are compressed and formed against the occlusal surfaces (212), and are substantially thinned or broken through at the regions adjacent to the protrusions (32, 34). However, since the thickness of a majority of the occlusal portions (64, 66) is maintained by the protrusions (32, 34), the posterior teeth (206, 208) are prevented from completely cutting through the occlusal portions (64, 66), which would ruin the oral appliance (22) by severely weakening or completely severing the terminal portions (50, 51) of the oral appliance (22).

The user should continue biting down on the oral appliance (22) held by the tray (20) for approximately 3 minutes or until the oral appliance (22) can substantially maintain its shape. The compressible member (24) is compressed by the palatal arch (204) under biting pressure, such that the palatal portion (68) of the oral appliance (22) is pushed against and conforms to the palatal arch (204). Upon curing, the palatal portion (68) is configured to apply a slight pressure on the greater palatine nerve or other portion of the palate. The user then removes the tray (20) and oral appliance (22) from the mouth, and submerges the oral appliance (22) in cool water. Room temperature water or cooler water would be best to harden the oral appliance (22); but the temperature can be higher than room temperature and substantially lower than the softening temperature. Once hardened or cured, the oral appliance (22) is separated from the tray (20).

Thus, the oral appliance (22) has been molded to closely fit the posterior teeth (206, 208), so that when fitted back into the mouth, the molded fit prevents dislodging of the oral appliance (22). In one embodiment, the palatal portion (68) preferably touches the palatal arch (204) to gently remind the user of its presence without being visible to others and to provide appetite suppressing characteristics.

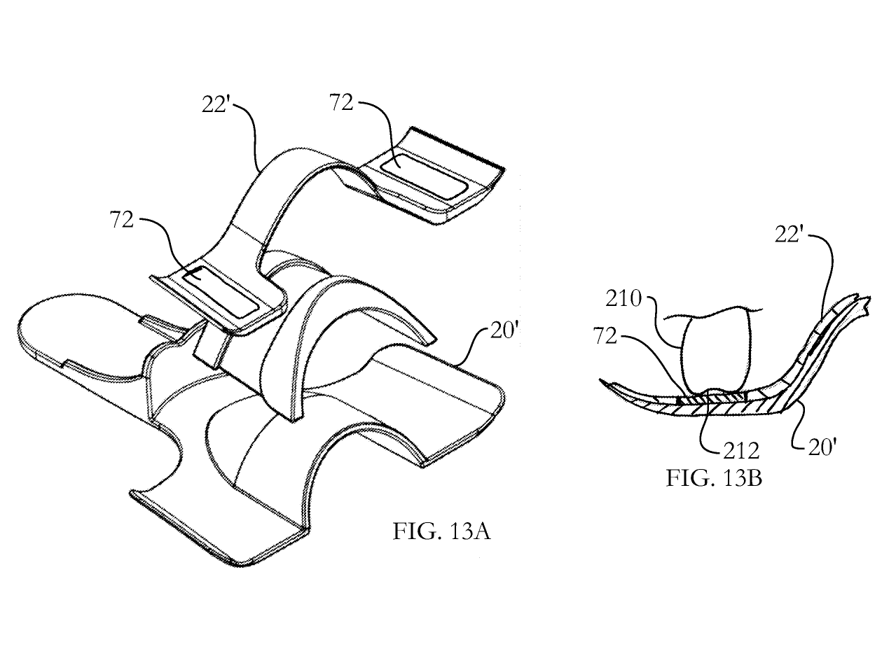

FIGS. 13A-B show an alternate embodiment of the present oral appliance (22′) and tray (20′) suitable for treatment of temporomandibular joint pain and dusfunction. At least a portion of the occlusal portion (64, 66) is made of a relatively hard material with a melting point that is higher than the thermosoftening plastic comprising the remainder of the oral appliance (22′) to form a unitary plural-material. The hard pad (72) is smooth and is configured to remain flat or only slightly curve while the user bites down during the curing process, such that the occlusal surface (212) does not deform the hard pad (72) nor leave a substantial impression. However, while soft, the terminal portions (50, 51) are still permitted to conform to the buccal sides (210); and the palatal portion (68) is still permitted to conform to the palatal arch (204). The hard pad (72) has a lower occlusal surface (74) that remains smooth. When fitted to the upper jaw (202), the occlusal surfaces of the lower jaw posterior teeth contact the lower occlusal surface (74) when the jaw is clenched, yet the lower jaw is free to move laterally relative to the upper jaw due to the lower jaw posterior teeth sliding freely on the lower occlusal surface (74).

The hard pad (72) can be inserted into a similarly shaped opening through the occlusal landing areas (26, 28), then held in place by adhesive, sonic or heat welded joint, press fit, or appropriate securing means. Alternatively, the hard pad (72) can be secured to the undersides (70) of the occlusal landing areas (26, 28). In this example, the occlusal surfaces (212) of the upper jaw posterior teeth (206, 208) may still contact and indent the softened occlusal portions (64, 66), but the lower occlusal surface (74) remains smooth to alleviate the effects of bruxing. The tray (20′) does not require the protrusions (32, 34), as it is a desire for the lower occlusal surfaces (74) to remain smooth, and the hard plastic cannot be easily bitten through.

Although the example embodiments of the present tray and oral appliance illustrate an appetite controlling device and an anti-bruxing device. The present tray can be used for fitting a wide variety of oral appliances, where a good fit is desired or when it is a desire to prevent the user form biting through the oral appliance while soft.

While particular forms of the invention have been illustrated and described, it will also be apparent to those skilled in the art that various modifications can be made without departing from the spirit and scope of the invention. Accordingly, it is not intended that the invention be limited except by the claims.

What is claimed is:

1. A tray for fitting an oral appliance within a mouth of a user, the mouth having a palatal arch and an upper jaw with a plurality of left posterior teeth and a plurality of right posterior teeth, the tray comprising:
a left occlusal landing area configured to receive the left posterior teeth;
a right occlusal landing area configured to receive the right posterior teeth;
a connecting portion spanning between the left occlusal landing area and the right occlusal landing area; and
a compressible member on the connecting portion;
wherein the oral appliance is overlaid atop the left occlusal landing area, the right occlusal landing area, and the compressible member which are cooperatively shaped to closely fit the oral appliance;
and wherein the oral appliance is configured to be positioned between the compressible member and the palatal arch while in a moldable state, and upon biting down the compressible member is compressed by the palatal arch with the oral appliance being formed against the palatal arch.

2. The tray of claim 1 wherein the compressible member is an arch.

3. The tray of claim 2 wherein a radius of the arch is configured to be smaller than the radius of the palatal arch of the user, such that compression of the compressible member causes the radius of the arch to deform to substantially the contours of the palatal arch.

4. The tray of claim 2 wherein a height of the arch is configured to be taller than the height of the palatal arch of the user, such that compression of the compressible member causes the height of the arch to deform to substantially the contours of the palatal arch.

5. The tray of claim 2 wherein a radius of the arch is configured to be smaller than the radius of the palatal arch of the user and a height of the arch is configured to be taller than the height of the palatal arch of the user, such that compression of the compressible member causes the arch to deform to the contours of the palatal arch.

6. The tray of claim 1 wherein the left occlusal landing area has a left upper surface and a left protrusion extending from the left upper surface and the right occlusal landing area has a right upper surface and a right protrusion extending from the right upper surface;
and wherein upon the user biting down upon the oral appliance while in a moldable state, the left protrusion is configured to prevent occlusal surfaces of the left posterior teeth from contacting the left upper surface, and the right protrusion is configured to prevent occlusal surfaces of the right posterior teeth from contacting the right upper surface.

7. The tray of claim 1 wherein a left flexible portion extends from a left buccal side of the left occlusal landing area and a right flexible portion extends from a right buccal side of the right occlusal landing area;
and wherein upon the user biting down upon the oral appliance while in a moldable state, the left flexible portion is configured to be flexed upwardly by contact with the left cheek and flexed towards the left buccal sides of the left posterior teeth, and the right flexible portion is configured to be flexed upwardly by contact with the right cheek and flexed towards the right buccal sides of the right posterior teeth.

8. The tray of claim 1 wherein the compressible member has a groove shaped to grip the oral appliance.

9. A tray for fitting an oral appliance within a mouth, the mouth having an upper jaw with a plurality of posterior teeth with each having a buccal side adjacent to a cheek, the tray comprising:
an occlusal landing area configured to receive the posterior teeth;
a flexible portion extending from the buccal side of the occlusal landing area; and
wherein the occlusal landing area is configured to support a portion of the oral appliance, where the portion is overlaid atop the occlusal landing area and the flexible portion;
and wherein upon the user biting down upon the oral appliance while in a moldable state, the flexible portion is configured to be flexed upwardly by contact with the cheek and flexed towards the buccal sides of the posterior teeth.

10. The tray of claim 9 further comprising:
a left occlusal landing area configured to receive the left posterior teeth;
a right occlusal landing area configured to receive the right posterior teeth;
a left flexible portion extending from a left buccal side of the left occlusal landing area; and
a right flexible portion extending from a right buccal side of the right occlusal landing area;
wherein the left occlusal landing area, the left flexible portion, the right occlusal landing area, and the right flexible portion are shaped to closely fit the oral appliance;
and wherein the left flexible portion and the right flexible portion are configured to be flexed upwardly by contact with the cheeks and flexed towards the buccal sides of the left posterior teeth and the right posterior teeth respectively.

11. The tray of claim 10 further comprising:
a connecting portion spanning between the left occlusal landing area and the right occlusal landing area; and
a compressible member on the connecting portion;
wherein upon biting down the compressible member is compressed by the palatal arch with the oral appliance being formed against the palatal arch.

12. The tray of claim 9 wherein the occlusal landing area has an upper surface and a protrusion extending from the upper surface; and upon the user biting down upon the oral appliance while in a moldable state, the protrusion on the upper surface is configured to prevent the occlusal surfaces of the posterior teeth from contacting the upper surface of the occlusal landing area.

13. The tray of claim 9 wherein the flexing of the flexible portion aids in forming the oral appliance against the buccal sides of the posterior teeth such that a terminal portion of the oral appliance extends over the height of contours of the posterior teeth and is formed to at least a portion of the infrabulge areas.

14. A tray for fitting an oral appliance within a mouth, the mouth having an upper jaw with a plurality of posterior teeth with each having an occlusal surface, the tray comprising:
an occlusal landing area with an upper surface and a protrusion extending from the upper surface, the occlusal landing area being configured to receive the posterior teeth;
wherein the occlusal landing area is configured to support a portion of the oral appliance, where the portion is overlaid atop the upper surface;
and wherein upon the user biting down upon the oral appliance while in a moldable state, the protrusion on the upper surface is configured to prevent the occlusal surfaces of the posterior teeth from contacting the upper surface of the occlusal landing area.

15. The tray of claim 14 further comprising:
a left occlusal landing area with a left upper surface with a left protrusion extending from the left upper surface; and
a right occlusal landing area with a right upper surface with a left protrusion extending from the left upper surface.

16. The tray of claim 14 wherein the protrusion is a ridge.

17. The tray of claim 14 wherein a plurality of protrusions extend from the upper surface.

18. The tray of claim 15 further comprising:
a connecting portion spanning between the left occlusal landing area and the right occlusal landing area; and
a compressible member on the connecting portion;
wherein upon biting down the compressible member is compressed by the palatal arch with the oral appliance being formed against the palatal arch.

19. The tray of claim 15 wherein a left flexible portion extends from a left buccal side of the left occlusal landing area and a right flexible portion extends from a right buccal side of the right occlusal landing area;
and wherein upon the user biting down upon the oral appliance while in a moldable state, the left flexible portion is configured to be flexed upwardly by contact with the left cheek and flexed towards the left buccal sides of the left posterior teeth, and the right flexible portion is configured to be flexed upwardly by contact with the right cheek and flexed towards the right buccal sides of the right posterior teeth.
